(19) United States
(12) Patent Application Publication
(10) Pub. No.: US 2021/0275738 A1
(43) Pub. Date: Sep. 9, 2021

(54) NPWT MODULE

(71) Applicant: Murata Manufacturing Co., Ltd., Kyoto (JP)

(72) Inventors: Susumu Takeuchi, Kyoto (JP); Kiyoshi Kurihara, Kyoto (JP)

(21) Appl. No.: 17/329,323

(22) Filed: May 25, 2021

Related U.S. Application Data
(63) Continuation of application No. PCT/JP2019/043257, filed on Nov. 5, 2019.

(30) Foreign Application Priority Data
Nov. 27, 2018 (JP) 2018-221007

(51) Int. Cl. A61M 1/00

(52) U.S. Cl. CPC A61M 1/962; A61M 2205/3331; A61M 2205/0294; A61M 2205/8206

(57) ABSTRACT

An NPWT module includes a suction module including a suction device that has an intake port, an exhaust port, and a suction main body connected to the intake port and the exhaust port, a power supply that supplies power to the suction device, and a control circuit that controls power supply performed by the power supply and a water-absorbing member having a flat shape, and the NPWT module has a first space in which the suction module is located and a second space that communicates with the intake port of the suction device and in which the water-absorbing member is located. The first space and the second space are positioned adjacent to each other.

### CROSS REFERENCE TO RELATED APPLICATION

This is a continuation of International Application No. PCT/JP2019/043257 filed on Nov. 5, 2019 which claims priority from Japanese Patent Application No. 2018-221007 filed on Nov. 27, 2018. The contents of these applications are incorporated herein by reference in their entireties.

### BACKGROUND OF THE DISCLOSURE

### Field of the Disclosure

The present disclosure relates to a negative pressure wound therapy (NPWT) module.

### Description of the Related Art

In the related art, an NPWT module that draws an exudate out of an affected area, which is a damaged portion of the skin such as a burn, a wound, or a bedsore, by maintaining negative pressure applied to the affected area has been designed. By applying negative pressure to an affected area, an exudate can be efficiently drawn out, and in addition, the affected area is dried, so that the treatment period is shortened.

For example, Patent Document 1 describes an NPWT module in which an absorbable layer accommodating a micropump system is disposed on a packing layer packed in a wound bed and in which the absorbable layer is covered from above with an upper layer.
Patent Document 1: Japanese Unexamined Patent Application Publication (Translation of PCT Application) No. 2009-506878

### BRIEF SUMMARY OF THE DISCLOSURE

However, in an NPWT module of the related art, the negative pressure that is created by a suction device causes an upper layer (a cover) covering the suction device to push down the suction device that is made of a metal or a resin toward a wound, and this irritates an affected area.

Accordingly, it is an object of the present disclosure to provide an NPWT module that is less irritating to an affected area.

To achieve the above-described object, an NPWT module according to the present disclosure includes a suction module including a suction device that has an intake port, an exhaust port, and a suction main body connected to the intake port and the exhaust port, a power supply that supplies power to the suction device, and a control circuit that controls power supply performed by the power supply and a water-absorbing member having a flat shape, and the NPWT module has a first space in which the suction main body is located and a second space that communicates with the intake port of the suction device and in which the water-absorbing member is located. The first space and the second space are positioned adjacent to each other.

According to the present disclosure, an NPWT module that is less irritating to an affected area can be provided.

### BRIEF DESCRIPTION OF THE SEVERAL VIEWS OF THE DRAWINGS

FIG. 1 is a diagram illustrating how to use an NPWT module in Embodiment 1.

FIG. 2A is a plan view of the NPWT module in Embodiment 1.

FIG. 2B is a longitudinal sectional view of the NPWT module taken along line IIb-IIb of FIG. 2A.

FIG. 2C is a longitudinal sectional view illustrating the NPWT module in FIG. 2B by omitting illustration of a water-absorbing member.

FIG. 3 is a block diagram of a suction module.

FIG. 4 is a plan view of an NPWT module in Embodiment 2.

FIG. 5A is a plan view of an NPWT module in Embodiment 3.

FIG. 5B is a longitudinal sectional view of the NPWT module taken along line Vb-Vb of FIG. 5A.

FIG. 6A is a plan view of an NPWT module in Embodiment 4.

FIG. 6B is a longitudinal sectional view of the NPWT module taken along line VIb-VIb of FIG. 6A.

FIG. 7 is a longitudinal sectional view of an NPWT module in Embodiment 5.

### DETAILED DESCRIPTION OF THE DISCLOSURE

An NPWT module according to an aspect of the present disclosure includes a suction module including a suction device that has an intake port, an exhaust port, and a suction main body connected to the intake port and the exhaust port, a power supply that supplies power to the suction device, and a control circuit that controls power supply performed by the power supply and a water-absorbing member having a flat shape, and the NPWT module has a first space in which the suction main body is located and a second space that communicates with the intake port of the suction device and in which the water-absorbing member is located. The first space and the second space are positioned adjacent to each other.

In such a configuration, negative pressure is created in the second space, which communicates with the intake port of the suction device, by the suction module. Thus, an exudate from an affected area is absorbed by the water-absorbing member located in the second space. In contrast, negative pressure is not applied to the first space, in which the suction main body is located, and thus, the probability that the suction main body will be pushed down toward the skin of a patient can be reduced, and irritation of the affected area can be prevented. In addition, since the first space, in which the suction main body is located, and the second space, in which the water-absorbing member is located, are adjacent to each other, the NPWT module is portable. Therefore, a patient can move even when the NPWT module is operating, and this can lighten the burden imposed on the patient.

In addition, the suction main body of the suction device may not overlap the water-absorbing member in a direction in which a main surface of the water-absorbing member is viewed in plan view. In such a configuration, when the affected area is put under negative pressure by the suction module, the suction main body of the suction device is located at a position that does not overlap the water-absorbing member, which is pressed against the affected area, when viewed in plan view, and thus, the suction main body can be prevented from being pressed against the affected area and further irritating the affected area.

In addition, the power supply and the control circuit of the suction module may not overlap the water-absorbing member in the direction in which the main surface of the water-absorbing member is viewed in plan view. In such a configuration, when the affected area is put under negative pressure by the suction module, the power supply and the control circuit can be prevented from being pressed against the affected area and further irritating the affected area.

In addition, the NPWT module may further include a cover that covers the first space and the second space. In such a configuration, the suction module located in the first space and the water-absorbing member located in the second space can be appropriately integrated with each other.

In addition, in the cover, an air permeability of a first portion that covers the second space may be lower than an air permeability of the second portion that covers the first space. In such a configuration, the air can be prevented from flowing into the water-absorbing member in the second space through the first portion of the cover, and the probability that the negative pressure applied to the water-absorbing member and the affected area will be released can be reduced.

In addition, the exhaust port may be open in the first space covered with the cover. In such a configuration, the air discharged from the exhaust port flows toward the suction module in the first space, and thus, the power supply and the control circuit of the suction module can be cooled.

In addition, the intake port and the exhaust port of the suction device may be open in a direction along the main surface of the water-absorbing member having a flat shape. In such a configuration, the intake port and the exhaust port of the suction device do not extend in a direction perpendicular to the main surface of the water-absorbing member having a flat shape, and thus, a reduction in the height of the NPWT module can be achieved.

In addition, the water-absorbing member may be located in the direction in which the intake port is open. By positioning the water-absorbing member in the direction in which the intake port is open, the pressure loss in the intake port can be reduced.

In addition, in the direction in which the main surface of the water-absorbing member is viewed in plan view, a region of the second space may be positioned so as to at least partially surround a region of the first space in which the suction module is located. In this configuration, the space around the periphery of the suction module is at least partially suctioned, and thus, the overall height of the NPWT module can be reduced. Furthermore, the suction module can be prevented from being greatly separated from the skin, and the probability that the suction device, the power supply, and the control circuit will be separated from the skin of the patient can be reduced.

In addition, the suction device may include a piezoelectric pump. In this configuration, the water-absorbing member and the affected area can be suitably put under a negative pressure lower than atmospheric pressure.

An NPWT module according to the present disclosure will be described below with reference to the drawings. Note that, in the drawings, members that have substantially the same functions or configurations are denoted by the same reference signs, and descriptions thereof may sometimes be omitted in the specification. In addition, for ease of understanding, the drawings schematically illustrate each component as a main component.

Note that the embodiments that will be described below are each a specific example of the present disclosure, and the present disclosure is not limited to the configurations described in the embodiments. In addition, numerical values, shapes, configurations, steps, the order of the steps, and so forth that are specifically described in the following embodiments are examples and are not intended to limit the scope of the present disclosure. Among the components in the following embodiments, the components that are not mentioned in the independent claim representing the most generic concept of the present disclosure will be described as arbitrary components. The same applies to modifications of all the embodiments, and the configurations described in the modifications may be combined with each other.

### Embodiment 1

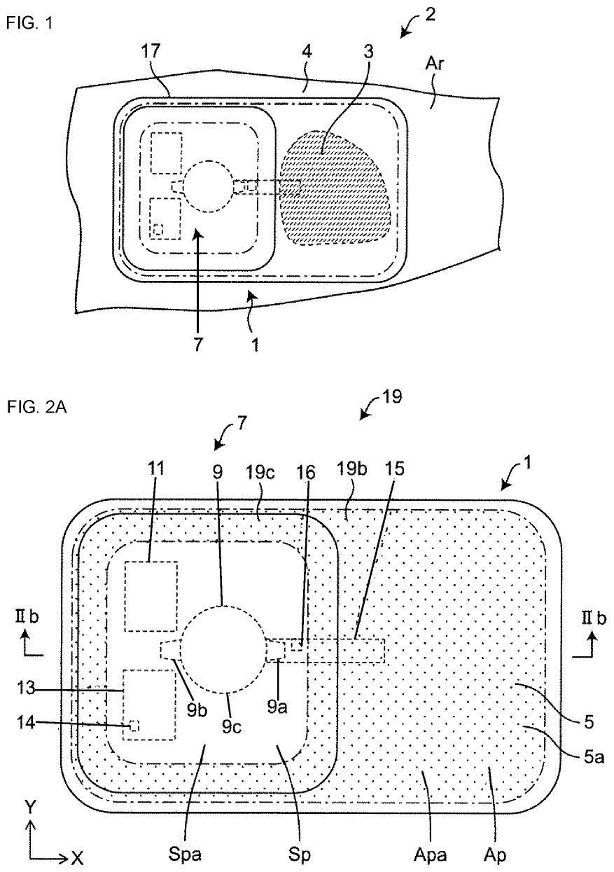

First, a usage pattern of an NPWT module 1 will be described with reference to FIG. 1. FIG. 1 is a diagram illustrating a state where the NPWT module 1 in Embodiment 1 is attached to an affected area 3 of a body surface Ar of a patient.

The NPWT module 1 is a portable module that is attachable to the affected area 3, which is a damaged portion of a skin 2. Unlike a stationary NPWT device, the NPWT module 1 enables a patient to freely move while the NPWT module 1 is drawing an exudate out of the affected area 3. In addition, the NPWT module 1 can be used at home.

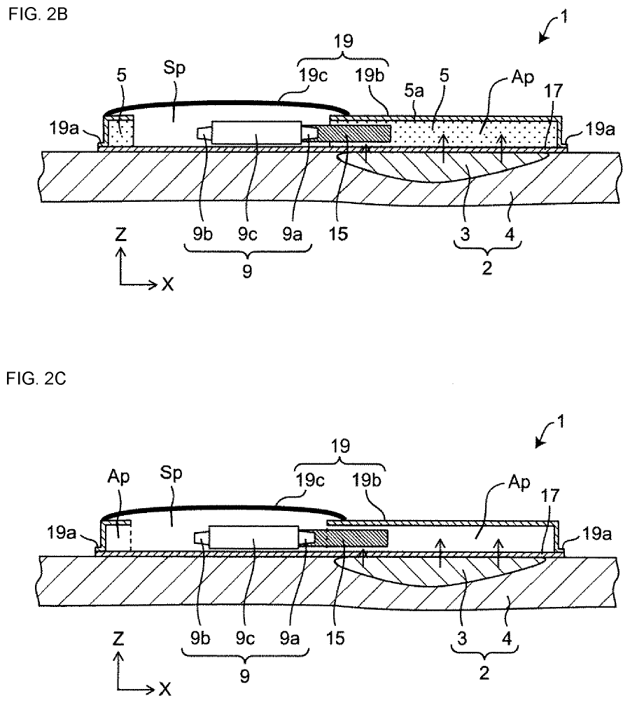

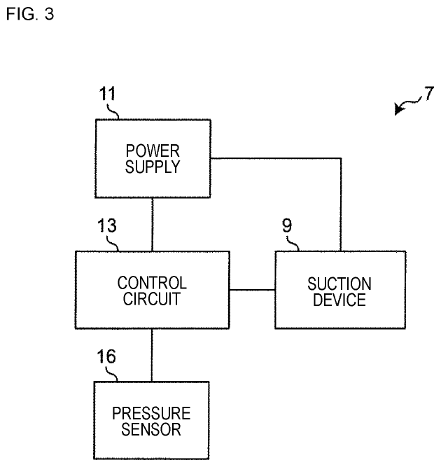

Next, the structure of the NPWT module 1 will be described with reference to FIG. 2A to FIG. 3. FIG. 2A is a plan view of the NPWT module in Embodiment 1. FIG. 2B is a longitudinal sectional view of the NPWT module taken along line IIb-IIb of FIG. 2A. FIG. 2C is a longitudinal sectional view illustrating the NPWT module in FIG. 2B by omitting illustration of a water-absorbing member. FIG. 3 is a block diagram illustrating a control configuration of a suction module. In the drawings that will be referred to in the following description, the X-Y-Z coordinate system is used for ease of understanding of the disclosure and is not intended to limit the scope of the disclosure. The X-axis direction corresponds to the longitudinal direction of the NPWT module 1. The Z-axis direction corresponds to a thickness direction (a height direction) of the NPWT module 1. The Y-axis direction is a direction perpendicular to the X-axis direction and the Z-axis direction. The X-axis direction, Y-axis direction, and Z-axis direction are orthogonal to one another.

### 1. NPWT Module

The NPWT module 1 includes a water-absorbing member 5 that absorbs an exudate from the affected area 3 and a suction module 7 that puts the affected area 3 and the water-absorbing member 5 under negative pressure lower than atmospheric pressure, and the NPWT module 1 has a first space Sp in which the suction module 7 is disposed and a second space Ap in which the water-absorbing member 5 is disposed. The suction module 7 and the water-absorbing member 5 are arranged on a placement surface of the NPWT module 1 that is placed onto the skin 2. In Embodiment 1, the placement surface of the NPWT module 1 that is placed onto the skin 2 is a main surface of a sheet 17 that is brought into contact with the skin 2. There is a normal skin portion or a non-affected area 4 that is in less need of treatment around and below the affected area 3.

The water-absorbing member 5 includes, for example, a piece of nonwoven fabric such as gauze, a piece of composite nonwoven fabric, a piece of cellulose woven fabric, a high-absorbent polymer, or a combination of these. The water-absorbing member 5 has a flat shape and communicates with an intake port 9a of a suction device 9. The water-absorbing member 5 is disposed so as to fill the second space Ap.

The suction module 7 includes the suction device 9 that has the intake port 9a, an exhaust port 9b, and a suction main body 9c connected to the intake port 9a and the exhaust port 9b, a power supply 11 that supplies power to the suction device 9, and a control circuit 13 that controls power supply performed by the power supply 11. Each of the intake port 9a and the exhaust port 9b may have a nozzle shape as illustrated in the drawings or may simply have a hole shape.

In Embodiment 1, as an example in which the intake port 9a of the suction device 9 and the second space Ap communicate with each other, the water-absorbing member 5, which is disposed so as to fill the second space Ap, and the intake port 9a of the suction device 9 communicate with each other via a pipe 15. The state in which the pipe 15 communicates with the water-absorbing member 5 may be a state in which the pipe 15 is inserted in the water-absorbing member 5 or a state in which an end of the pipe 15 is in contact with the water-absorbing member 5. As a result of the pipe 15 communicating with the water-absorbing member 5, the water-absorbing member 5 is put under negative pressure when the suction device 9 operates. The pipe 15 is made of, for example, a flexible material made of a resin, and thus, even if the water-absorbing member 5 is pushed toward the affected area 3 as a result of being put under negative pressure, irritation of the affected area 3 is reduced. Note that the intake port 9a, having a nozzle shape, of the suction device 9 may directly communicate with the water-absorbing member 5. In addition, a filter that allows air to pass therethrough while not allowing moisture to pass therethrough is disposed in the pipe 15 or the intake port 9a. As a result, the moisture absorbed by the water-absorbing member 5 can be prevented from flowing into the suction main body 9c of the suction device 9.

The suction device 9 includes, for example, a piezoelectric pump or a small-sized motor pump. Each of the intake port 9a and the exhaust port 9b of the suction device 9 is open in a direction (X-axis direction) along a placement surface of the skin 2. In addition, the water-absorbing member 5 that is placed onto the affected area 3 is located in the direction in which the intake port 9a is open. The suction main body 9c has a pump function of drawing in the air.

In the suction device 9, although the intake port 9a may overlap the water-absorbing member 5, the suction main body 9c does not overlap the water-absorbing member 5 in a direction in which a main surface 5a of the water-absorbing member 5 is viewed in plan view. In addition, the power supply 11 and the control circuit 13 of the suction module 7 do not overlap the water-absorbing member 5 in the direction in which the main surface 5a of the water-absorbing member 5 is viewed in plan view.

The power supply 11 is, for example, a battery. The battery may be a primary battery or a secondary battery.

The control circuit 13 includes an on/off switch 14 for starting and stopping decompression control, and when the on/off switch 14 is pushed by a user, power is supplied to the control circuit 13 and the suction device 9 from the power supply 11.

A pressure sensor 16 is disposed in the pipe 15. A value that is detected by the pressure sensor 16 is transmitted to the control circuit 13. The control circuit 13 controls the amount of power that is supplied to the suction device 9 from the power supply 11 on the basis of the value detected by the pressure sensor 16 such that the detected value becomes a predetermined value. The pressure value in the second space Ap in which the water-absorbing member 5 is located and the pressure value in a space in which the affected area 3 is located are each reduced to a value that is lower than atmospheric pressure by, for example, about 10 kpa. The method for controlling the amount of power may be on/off control or may be PWM control. In the case where the control circuit 13 performs on/off control, for example, when the detected value is equal to or lower than a value that is lower than atmospheric pressure by 12 kpa, power supply is stopped, and when the detected value is equal to or higher than a value that is lower than atmospheric pressure by 8 kpa, power supply is started so as to decompress the second space Ap, in which the water-absorbing member 5 is located, and the space in which the affected area 3 is located.

The NPWT module 1 further includes the sheet 17 that is attached to the affected area 3 and the skin 2 around the affected area 3, and a cover 19 that covers the water-absorbing member 5 and the suction module 7.

The water-absorbing member 5 and the suction module 7 are arranged on the upper surface of the sheet 17. In the sheet 17, at least a portion that is in contact with the water-absorbing member 5 has water permeability and is, for example, porous. In Embodiment 1, the entire surface of the sheet 17 has water permeability, and the sheet 17 is a film whose surface that is brought into contact with the skin 2 has a viscosity. Thus, the NPWT module 1 can be attached to the affected area 3 and the skin 2 around the affected area 3.

The cover 19 has an outer edge portion 19a that is bonded to the sheet 17. The cover 19 includes a first portion 19b that covers the water-absorbing member 5 and a second portion 19c that covers the suction module 7. The air permeability of the first portion 19b of the cover 19 is lower than the suction amount (the amount of ventilation) of the suction device 9, and thus, the second space Ap can be decompressed. In addition, the amount of the air that flows into the second space Ap can be reduced, and the amount of the air that flows into the water-absorbing member 5 and the affected area 3 via the first portion 19b can be reduced. In contrast, the air permeability of the second portion 19c covering the suction module 7 is higher than that of the first portion 19b covering the water-absorbing member 5, and thus, the first space Sp is less likely to be decompressed. Thus, the probability that the suction module 7 located in the first space Sp will be pressed against the skin of a patient can be reduced.

In addition, since the second portion 19c of the cover 19 has air permeability, the air that is discharged from the exhaust port 9b of the suction device 9 can be discharged to the outside through the second portion 19c. The first portion 19b and the second portion 19c may be formed of films that are different members.

The NPWT module 1 has two spaces, which are the second space Ap in which the water-absorbing member 5 is disposed and the first space Sp in which the suction module 7 is disposed. The first portion 19b of the cover 19 covers the second space Ap, and the second portion 19c covers the first space Sp. The first space Sp is formed by being surrounded by the second portion 19c of the cover 19, the sheet 17, and the water-absorbing member 5. The exhaust port 9b of the suction device 9 is open in the first space Sp. Thus, the air discharged from the exhaust port 9b flows in the first space Sp.

When viewed in plan view, a second region Apa on which the second space Ap, which is filled with the water-absorbing member 5, is projected is arranged so as to surround a first region Spa on which the first space Sp, in which the suction module 7 is located, is projected. As a result, during the operation of the suction module 7, the peripheral portion of the first region Spa can be brought into close contact with the skin 2 of the patient. Note that the second region Apa, in which the water-absorbing member 5 is disposed, may be arranged so as to at least partially surround the first region Spa, in which the suction module 7 is located, when viewed in plan view. The region in which the water-absorbing member 5 is disposed is a region to be decompressed, and thus, the degree of contact with the skin 2 of the patient can be increased as the area of the region in which the water-absorbing member 5 is disposed increases.

An operation when the NPWT module 1 is attached to the affected area 3 will now be described.

In the NPWT module 1, electrical power is supplied to the control circuit 13 and the suction device 9 when the on/off switch 14 is pushed by a user. The control circuit 13 controls, on the basis of the value detected by the pressure sensor 16, the amount of power that is supplied to the suction device 9. As a result, the amount of decompression that is performed by the suction device 9 can be controlled.

The space which is located in the place which is in contact the affected area 3 via the water-absorbing member 5 and the sheet 17 is decompressed, and an exudate from the affected area 3 passes through the sheet 17 and is absorbed by the water-absorbing member 5. In this case, the pressure in the first space Sp is the same as or slightly higher than atmospheric pressure, and thus, the pressure is higher than that in the water-absorbing member 5. Consequently, the exudate absorbed by the water-absorbing member 5 can be prevented from flowing into the first space Sp, and a malfunction of the suction module 7 can be prevented from occurring.

As described above, the NPWT module 1 of Embodiment 1 includes the suction module 7 including the suction device 9, which has the intake port 9a, the exhaust port 9b, and the suction main body 9c connected to the intake port 9a and the exhaust port 9b, the power supply 11, which supplies power to the suction device 9, and the control circuit 13, which controls power supply performed by the power supply 11, and the water-absorbing member 5, which has a flat shape and which is located in the second space Ap, and the NPWT module 1 has the first space Sp, in which the suction main body 9c is located, and the second space Ap, which communicates with the intake port 9a of the suction device 9. The first space Sp and the second space Ap are positioned adjacent to each other. Thus, negative pressure is created in the second space Ap, which communicates with the intake port 9a of the suction device 9, by the suction module 7. Consequently, an exudate from the affected area 3 is absorbed by the water-absorbing member 5 located in the second space Ap. In contrast, since the first space Sp, in which the suction main body 9c is located, is not decompressed, the probability that the suction main body 9c will be pushed down toward the skin 2 of the patient can be reduced, and irritation of the affected area 3 can be prevented. In addition, since the first space Sp, in which the suction main body 9c is located, and the second space Ap, in which the water-absorbing member 5 is located, are adjacent to each other, the NPWT module 1 is portable. Therefore, the patient can move even when the NPWT module 1 is operating, and this can lighten the burden imposed on the patient.

The suction main body 9c of the suction device 9 does not overlap the water-absorbing member 5 in the direction in which the main surface 5a of the water-absorbing member 5 is viewed in plan view. Accordingly, when the affected area 3 is put under negative pressure by the suction module 7, the suction main body 9c of the suction device 9 is located at a position that does not overlap the water-absorbing member 5, which is pressed against the affected area 3, when viewed in plan view, and thus, the suction main body 9c can be prevented from being pressed against the affected area 3 and further irritating the affected area 3.

The power supply 11 and the control circuit 13 of the suction module 7 do not overlap the water-absorbing member 5 in the direction in which the main surface 5a of the water-absorbing member 5 is viewed in plan view. Accordingly, when the affected area 3 is put under negative pressure by the suction module 7, the power supply 11 and the control circuit 13 of the suction module 7 are located at positions that do not overlap the water-absorbing member 5, which is pressed against the affected area 3, when viewed in plan view, and thus, the power supply 11 and the control circuit 13 can be prevented from being pressed against the affected area 3 and further irritating the affected area 3.

In addition, the NPWT module 1 includes the cover 19 that covers the first space Sp and the second space Ap. As a result, the suction module 7 and the water-absorbing member 5 can be appropriately integrated with each other.

In the cover 19, the air permeability of the first portion 19b covering the second space Ap is lower than that of the second portion 19c covering the first space Sp. As a result, the air can be prevented from flowing into the water-absorbing member 5 through the first portion 19b, and the probability that the negative pressure applied to the water-absorbing member 5 and the affected area 3 will be released can be reduced.

In addition, the exhaust port 9b is open in the first space Sp, which is covered with the cover 19. As a result, the air discharged from the exhaust port 9b flows in the first space Sp, and thus, the suction main body 9c of the suction device 9 can be cooled, so that a malfunction can be prevented from occurring. Since the suction module 7 is disposed in the first space Sp, the power supply 11 and the control circuit 13 of the suction module 7 can also be cooled, so that a malfunction of the power supply 11 and a malfunction of the control circuit 13 can also be prevented from occurring.

Each of the intake port 9a and the exhaust port 9b of the suction device 9 is open in the direction along the main surface 5a of the water-absorbing member 5 having a flat shape. As a result, the height of the NPWT module 1 can be further reduced.

The water-absorbing member 5 is located in the direction in which the intake port 9a is open. Accordingly, since the water-absorbing member 5 is located on an extension line that extends in the direction in which the intake port 9a is open, the pressure loss between the water-absorbing member 5 and the suction device 9 can be reduced.

The second region Apa of the second space Ap is disposed so as to at least partially surround the first region Spa of the first space Sp, in which the suction main body 9c is located, in the direction in which the main surface 5a of the water-absorbing member 5 is viewed in plan view. Accordingly, the water-absorbing member 5 at least partially surrounds the suction main body 9c, and thus, the peripheral portion of the suction main body 9c is suctioned, so that the height of the entirety of the NPWT module 1 can be reduced. In addition, since the peripheral portion of the suction module 7 is suctioned in the case where the suction module 7 is disposed in the first space Sp, the suction module 7 can be prevented from being greatly separated from the skin 2, and the probability that the suction device 9, the power supply 11, and the control circuit 13 will be separated from the skin 2 of the patient can be reduced.

### Embodiment 2

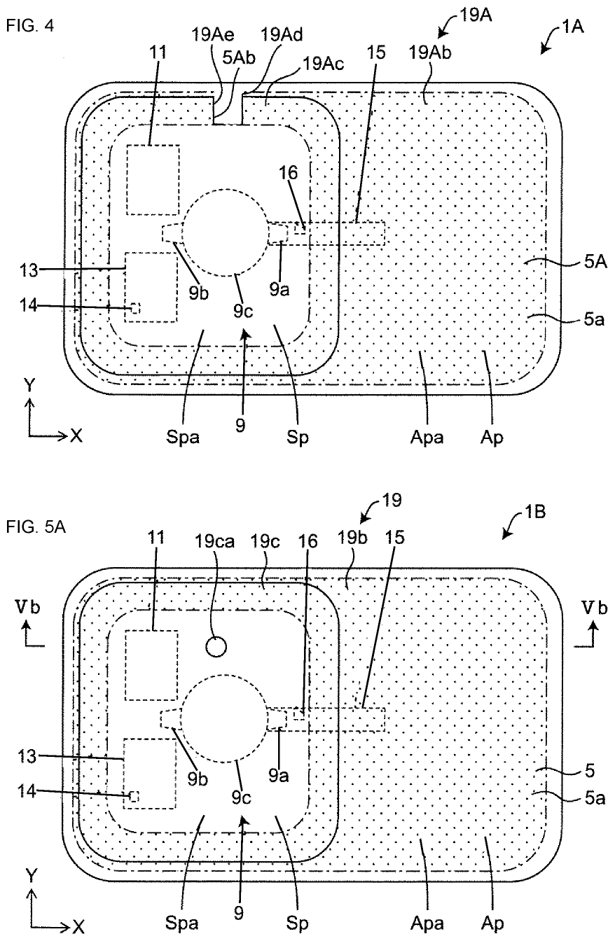

Next, an NPWT module 1A in Embodiment 2 will be described with reference to FIG. 4. FIG. 4 is a plan view of the NPWT module 1A in Embodiment 2.

The NPWT module 1A in Embodiment 2 has a configuration in which the second space Ap, which surrounds the first space Sp, the water-absorbing member 5, and the second portion 19c of the cover 19 in the NPWT module 1 in Embodiment 1 are partially missing. Note that, the configuration of the NPWT module 1A in Embodiment 2, excluding the matters described below, is the same as that of the NPWT module 1 of Embodiment 1.

A water-absorbing member 5A in Embodiment 2 has a portion that surrounds the first region Spa, and this portion has a cutout portion 5Ab. In addition, a first portion 19Ab of a cover 19A in Embodiment 2 also has a cutout portion 19Ad at the same position as the cutout portion 5Ab of the water-absorbing member 5A. Furthermore, a second portion 19Ac of the cover 19A in Embodiment 2 also has a cutout portion 19Ae at the same position as the cutout portion 5Ab of the water-absorbing member 5A. Accordingly, the cutout portion 5Ab of the water-absorbing member 5A and the cutout portions 19Ad and 19Ae of the cover 19A form a communication hole that allows communication between the first space Sp and the outside. As a result, the pressure in the first space Sp can be set to be the same as or close to atmospheric pressure with certainty. Thus, similar to the first portion 19Ab, the second portion 19Ac of the cover 19A can be formed of a film having a low air permeability, and the first portion 19Ab and the second portion 19Ac can also be formed of the same film.

### Embodiment 3

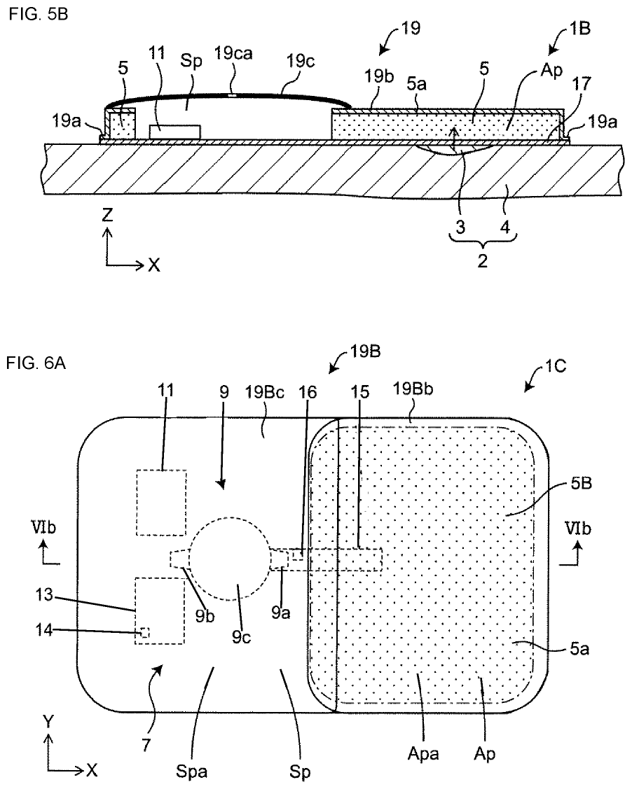

Next, an NPWT module 1B in Embodiment 3 will be described with reference to FIG. 5A and FIG. 5B. FIG. 5A is a plan view of the NPWT module 1B in Embodiment 3. FIG. 5B is a longitudinal sectional view of the NPWT module 1B taken along line Vb-Vb of FIG. 5A.

The NPWT module 1B in Embodiment 3 has a configuration in which a through hole 19ca is formed in the second portion 19c of the cover 19 of the NPWT module 1 in Embodiment 1. Note that, the configuration of the NPWT module 1B in Embodiment 3, excluding the matters described below, is the same as that of the NPWT module 1 of Embodiment 1.

The second portion 19c of the cover 19 in Embodiment 3 has the through hole 19ca that allows communication between the first space Sp and the outside. As a result, the pressure in the first space Sp can be set to be the same as or close to atmospheric pressure with certainty. Thus, similar to the first portion 19b, the second portion 19c of the cover 19 can be formed of a film having a low air permeability, and the first portion 19b and the second portion 19c of the cover 19 can also be formed of the same film.

### Embodiment 4

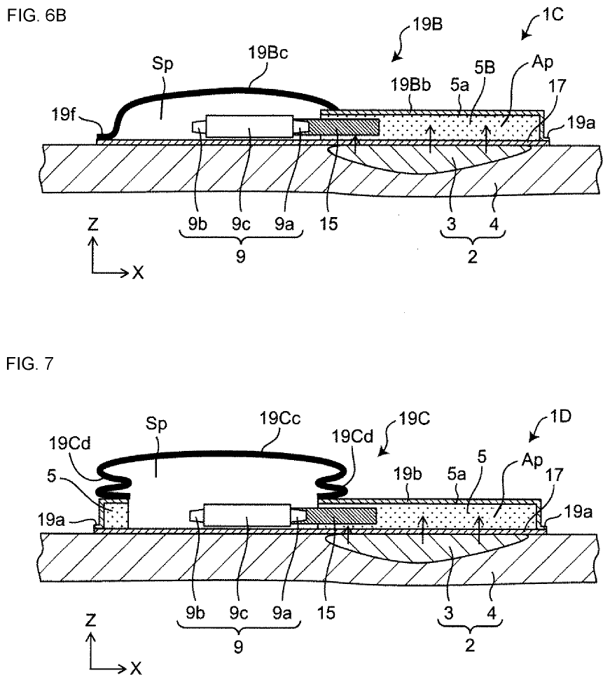

Next, an NPWT module 1C in Embodiment 4 will be described with reference to FIG. 6A and FIG. 6B. FIG. 6A is a plan view of the NPWT module 1C in Embodiment 4. FIG. 6B is a longitudinal sectional view of the NPWT module 1C taken along line VIb-VIb of FIG. 6A.

In the NPWT module 1C in Embodiment 4, although the water-absorbing member 5B is disposed on a placement surface of the affected area 3, the water-absorbing member 5B is not disposed so as to surround the suction module 7 unlike the water-absorbing member 5B in the NPWT module 1 in Embodiment 1. In other words, the second space Ap in which the water-absorbing member 5B is located is not formed so as to surround the first space Sp in which the suction module 7 is located. Note that, the configuration of the NPWT module 1C in Embodiment 4, excluding the matters described below, is the same as that of the NPWT module 1 of Embodiment 1.

A water-absorbing member 5B of Embodiment 4 is disposed in a region on one side in the longitudinal direction of the NPWT module 1C (X-axis direction). Thus, the water-absorbing member 5B is disposed on the placement surface of the affected area 3. A second portion 19Bc of a cover 19B has a substantially rectangular shape, and one side of the second portion 19Bc is connected to the first portion 19Bb. The other three sides of the second portion 19Bc are connected to the sheet 17 at an outer edge portion 19f. As described above, even in the case of employing the configuration in which the NPWT module 1C is divided into the first space Sp, in which the suction module 7 is located, and the second space Ap, in which the water-absorbing member 5 is located, in the longitudinal direction, since the suction module 7 is not disposed on the placement surface of the affected area 3, the suction module 7 will not press the affected area 3 even in the negative-pressure state, and irritation of the affected area 3 can be reduced.

### Embodiment 5

Next, an NPWT module 1D in Embodiment 5 will be described with reference to FIG. 7. FIG. 7 is a longitudinal sectional view of the NPWT module 1D in Embodiment 5.

The NPWT module 1D in Embodiment 5 has a configuration in which the second portion 19c of the cover 19 of the NPWT module 1 in Embodiment 1 has a connecting portion that is connected to the first portion 19b and folded. Note that, the configuration of the NPWT module 1D in Embodiment 5, excluding the matter described below, is the same as that of the NPWT module 1 of Embodiment 1.

In a second portion 19Cc of a cover 19C in Embodiment 5, a connecting portion that is connected to the first portion 19b has a folded portion 19Cd. By providing the folded portion 19Cd, even in the case where the second portion 19Cc of the cover 19C does not have stretchability and where the water-absorbing member 5 in the second space Ap is suctioned and flattened, the cover 19C will not be pulled such that the suction module 7 including the suction device 9 is pushed down.

The present disclosure is applicable to an NPWT module that maintains negative pressure applied on an affected area.
1, 1A, 1B, 1C, 1D NPWT module
2 skin
3 affected area
4 non-affected area
5, 5A, 5B water-absorbing member
5a main surface
5Ab cutout portion
7 suction module
9 suction device
9a intake port
9b exhaust port
9c suction main body
11 power supply
13 control circuit
14 on/off switch
15 pipe
16 pressure sensor
17 sheet
19, 19A, 19B, 19C cover
19a outer edge portion
19b, 19Ab, 19Cb first portion
19c, 19Ac, 19Cc second portion
19ca through hole
19Ad cutout portion
19Cd folded portion
Ap second space
Apa second region
Ar body surface
Sp first space
Spa first region

1. A negative pressure wound therapy module comprising:
a suction module including a suction device, a power supply, and a control circuit, wherein the suction device has an intake port, an exhaust port, and a suction main body connected to the intake port and the exhaust port, wherein the power supply supplies power to the suction device, and wherein the control circuit controls the power supplied by the power supply; and
a water-absorbing member having a flat shape,
wherein the negative pressure wound therapy module has a first space and a second place, wherein the suction main body is located in the first place, and wherein the second space communicates with the intake port of the suction device, and the water-absorbing member is located in the second place, and
wherein the first space and the second space are positioned adjacent to each other.

2. The negative pressure wound therapy module according to claim 1,
wherein the suction main body of the suction device does not overlap the water-absorbing member in a direction in which a main surface of the water-absorbing member is viewed in plan view.

3. The negative pressure wound therapy module according to claim 2,
wherein the power supply and the control circuit of the suction module do not overlap the water-absorbing member in the direction in which the main surface of the water-absorbing member is viewed in plan view.

4. The negative pressure wound therapy module according to claim 1, further comprising:
a cover covering the first space and the second space.

5. The negative pressure wound therapy module according to claim 4,
wherein the cover includes a first portion covering the second space and a second portion covering the first space, and wherein an air permeability of the first portion is lower than an air permeability of the second portion.

6. The negative pressure wound therapy module according to claim 4,
wherein the exhaust port is open in the first space covered with the cover.

7. The negative pressure wound therapy module according to claim 1,
wherein the intake port and the exhaust port of the suction device are open in a direction along a main surface of the water-absorbing member having a flat shape.

8. The negative pressure wound therapy module according to claim 1,
wherein the water-absorbing member is located in a direction in which the intake port is open.

9. The negative pressure wound therapy module according to claim 1,
wherein, in a direction in which a main surface of the water-absorbing member is viewed in plan view, a region of the second space is positioned so as to at least partially surround a region of the first space in which the suction module is located.

10. The negative pressure wound therapy module according to claim 1,
wherein the suction device includes a piezoelectric pump.

11. The negative pressure wound therapy module according to claim 2, further comprising:
a cover covering the first space and the second space.

12. The negative pressure wound therapy module according to claim 3, further comprising:
a cover covering the first space and the second space.

13. The negative pressure wound therapy module according to claim 5,
wherein the exhaust port is open in the first space covered with the cover.

14. The negative pressure wound therapy module according to claim 2,
wherein the intake port and the exhaust port of the suction device are open in a direction along a main surface of the water-absorbing member having a flat shape.

15. The negative pressure wound therapy module according to claim 3,
wherein the intake port and the exhaust port of the suction device are open in a direction along a main surface of the water-absorbing member having a flat shape.

16. The negative pressure wound therapy module according to claim 4,
wherein the intake port and the exhaust port of the suction device are open in a direction along a main surface of the water-absorbing member having a flat shape.

17. The negative pressure wound therapy module according to claim 5,
wherein the intake port and the exhaust port of the suction device are open in a direction along a main surface of the water-absorbing member having a flat shape.

18. The negative pressure wound therapy module according to claim 6,
wherein the intake port and the exhaust port of the suction device are open in a direction along a main surface of the water-absorbing member having a flat shape.

19. The negative pressure wound therapy module according to claim 2,
wherein the water-absorbing member is located in a direction in which the intake port is open.

20. The negative pressure wound therapy module according to claim 3,
wherein the water-absorbing member is located in a direction in which the intake port is open.
